(19) United States
(12) Patent Application Publication
(10) Pub. No.: US 2009/0198199 A1
(43) Pub. Date: Aug. 6, 2009

(54) INERT CONTAINER AND DISPENSER

(75) Inventors: Jacob Griggs, Union, KY (US); Joel Ivers, Cincinnati, OH (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: DRUG ENHANCEMENT COMPANY OF AMERICA, LLC, Union, KY (US)

(21) Appl. No.: 12/024,358

(22) Filed: Feb. 1, 2008

(51) Int. Cl. A61M 35/00

(52) U.S. Cl. 604/290; 604/311

(57) ABSTRACT

The invention relates to devices and methods for containing and dispensing a medicant, pharmaceutical product or other agent. Various elements of the devices may be constructed of materials that do not have a deleterious impact on the chemical composition, medicinal efficacy or other properties of the agent. In one embodiment, the device includes a vial with a pump sprayer, configured to contain a solution of oxychlorine compounds. The pump sprayer may be fitted with an insert constructed of glass, borosilicate glass, PTFE, PFA, chemically-modified PTFE or another inert material, and the entire assembly may be housed within a barrel and cap. Methods of using the aforementioned device to prevent, treat and/or reduce the risk of an infection on a surface area are also described.

### FIELD OF THE INVENTION

The invention relates to the field of containers and dispensers for a variety of compositions.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

There are countless pharmaceuticals, medicants and other agents that are or may be used by consumers, patients, physicians, heath care workers, paramedics, first responders, military personnel and emergency workers. A variety of storage systems are available in the art for these agents, and may be used by an individual to transport and/or administer a small quantity of the agent. For instance, a small bottle with a pump sprayer and cap might be used to transport a volume of a therapeutic agent that can be sprayed in a person's mouth.

Selection of a storage system for a particular agent may be based on a variety of factors, such as the durability, cost, ease of manufacturing or functionality of the storage system. However, the chemical properties of the agent and the relationship of those properties to the storage system must also be considered. So, too, must one consider the setting in which the storage system will be utilized.

Some agents can be stored in any number of storage systems, because their chemical properties are such that the agents do not substantially degrade within, permeate through, react with or otherwise experience deleterious effects as a result of the storage system; or, more particularly, as a result of a chemical interaction with the materials used to construct the storage system. Other agents are more discriminating. They might become altered, seep out of, interact with or otherwise be affected by the storage system material in such a manner that the agents lose their medicinal efficacy or the like. Thus, particularly in connection with pharmaceutical and medicinal agents, care must be exercised in selecting an appropriate storage system and materials to construct the same.

Regarding the setting in which a storage system may be used, it might not be desirable to use, for example, a storage system constructed of a material that is easily breakable when the system is to be carried by an emergency worker in the field. There, the performance of even routine tasks may compromise the integrity of the storage system. It might become inoperative or shatter and spill its contents.

Storage and delivery systems currently available in the art that enable individuals to conveniently transport small quantities of pharmaceuticals, medicants and other agents are frequently constructed from plastic, rubber and/or similar materials. However, as described above, many agents cannot be stored in such containers without losing efficacy or experiencing other undesirable effects. Moreover, the environment in which such storage and delivery systems may be used necessitates, in some cases, the use of a system that can withstand the exertion of anything from minor jostling to severe blunt force.

There is therefore a need in the art for a storage and delivery system that overcomes the aforementioned challenges.

### SUMMARY OF THE INVENTION

In one embodiment, the invention includes an apparatus, comprising: a vial to contain an agent; and a dispenser assembly to dispense the agent from the vial, the dispenser assembly mechanically affixed to the vial and including elements that come into contact with the agent irrespective of actuation, wherein the vial and the elements that come into contact with the agent irrespective of actuation consist essentially of materials that are inert or non-reactive with the agent. The apparatus may further include a barrel and cap, within which the vial and dispenser assembly are fitted. The apparatus may further comprise a quantity of an agent. The agent may be a composition to prevent and/or treat and/or reduce the risk of an infection. The agent may contain about 99.99% oxidized water, sodium hypochlorite (NaOCl), hypochlorous acid (HOCl) and sodium chloride (NaCl). The barrel may further comprise a series of flanges to retain the vial within the barrel. The series of flanges may be configured to separate from one another to allow the vial to be inserted into the barrel, and thereafter substantially assume an initial shape that retains the vial within the barrel. The barrel may further comprise at least one support element in mechanical contact with the vial. The dispenser assembly may be a pump sprayer. The pump sprayer may further comprise an insert portion extending axially from the portion of the pump sprayer that remains external to the vial, into an interior of the vial. The insert portion may be a unitary item. The vial and the insert portion may each be constructed of materials independently selected from the group consisting of glass, borosilicate glass, PTFE, PFA, chemically-modified PTFE, inert materials and combinations thereof. The apparatus may further comprise a washer positioned between the vial and the dispenser assembly to provide and/or enhance a fluid seal therebetween. The washer may be constructed of a material selected from the group consisting of PTFE, PFA, chemically-modified PTFE, inert materials and combinations thereof. The vial and the dispenser assembly may be affixed to one another with a mechanism selected from the group consisting of screw threading, a snap fitting, a pressure fitting, a heat seal, a chemical adhesive and combinations thereof. The series of flanges may each comprise a locking element configured to mechanically interact with at least one receiving element configured on an interior surface of the cap. The cap may further comprise a tab configured to allow a user to remove the cap from the barrel by dislodging the locking elements from the at least one receiving element. The tab may comprise a forward portion affixed to a side of the cap, a rear portion configured to receive a mechanical force from the user to separate the cap from the barrel, and an intermediate portion therebetween. The angle between the forward portion and an axis of the cap may be from about 0° to about 10°, the angle between the intermediate portion and the axis of the cap may be from about 45° to about 55°, and the angle between the rear portion and the axis of the cap may be from about 0° to about 8°. The rear portion may comprise a surface configured to receive a finger of the user and, upon application of a generally radial force thereupon, dislodge the cap from the barrel.

In another embodiment, the invention includes a method of preventing and/or treating and/or reducing the risk of an infection in a subject in need thereof, comprising: providing an apparatus, comprising a vial containing a composition to prevent and/or treat and/or reduce the risk of an infection, a dispenser assembly to dispense the composition from the vial, the dispenser assembly mechanically affixed to the vial and including elements that come into contact with the composition irrespective of actuation, and a barrel and cap, within which the vial and the dispenser assembly are fitted, wherein the vial and the elements that come into contact with the composition irrespective of actuation consist essentially of materials that are inert or non-reactive with the composition; removing the cap from the barrel; and dispensing the composition onto a surface of the subject to prevent and/or treat and/or reduce the risk of the infection. The composition may contain about 99.99% oxidized water, sodium hypochlorite (NaOCl), hypochlorous acid (HOCl) and sodium chloride (NaCl). The barrel may further comprise a series of flanges to retain the vial within the barrel, and the series of flanges may be configured to separate from one another to allow the vial to be inserted into the barrel, and thereafter substantially assume an initial shape that retains the vial within the barrel. The dispenser assembly may be a pump sprayer, comprising an insert portion extending axially from the portion of the pump sprayer that remains external to the vial, into an interior of the vial, and the insert portion may be a unitary item. The vial and the insert portion may each be constructed of materials independently selected from the group consisting of glass, borosilicate glass, PTFE, PFA, chemically-modified PTFE, inert materials and combinations thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts a perspective view of a vial and dispenser assembly in accordance with an embodiment of the present invention.

FIG. 2 depicts an exploded, perspective view of the vial and dispenser assembly shown in FIG. 1, in accordance with an embodiment of the present invention.

FIG. 3 depicts a cross-sectional view of the vial and dispenser assembly shown in FIG. 1, in accordance with an embodiment of the present invention.

FIG. 4 depicts a perspective view of the vial and dispenser assembly shown in FIG. 1 in combination with a barrel and cap (the cap is in the open position), in accordance with an embodiment of the present invention.

FIG. 5 depicts an exploded, perspective view of the vial and dispenser assembly with the barrel and cap shown in FIG. 4, in accordance with an embodiment of the present invention.

FIG. 6 depicts a cross-sectional view of the vial and dispenser assembly with the barrel and cap (the cap is in the closed position) shown in FIG. 4, in accordance with an embodiment of the present invention.

FIG. 7 depicts a top perspective view of a barrel, in accordance with an embodiment of the present invention.

FIG. 8 depicts a bottom perspective view of a cap, in accordance with an embodiment of the present invention.

### DETAILED DESCRIPTION

One skilled in the art will recognize many methods and materials similar or equivalent to those described herein, which could be used in the practice of the present invention. Indeed, the present invention is in no way limited to the methods and materials described.

The invention relates to a container and dispenser for a composition. The composition may be a medicant, pharmaceutical product, cosmetic or personal care product (e.g., perfume, repellant, deodorant, antiperspirant, hairspray, sunscreen), household product, cleaner or any liquid, solution, dispersion, gel or other fluid for which it might be beneficial to contain within and/or dispense with a device with components that are substantially inert or non-reactive therewith. As such, those of skill in the art will recognize numerous fluids that may be used in connection with alternate embodiments of the present invention. As used herein, the term “agent” is meant to include any of the aforementioned items, and in various embodiments of the present invention, all or substantially all of the components of the container or dispenser that are in contact with the agent are constructed of an inert or otherwise non-reactive material, with respect to the agent.

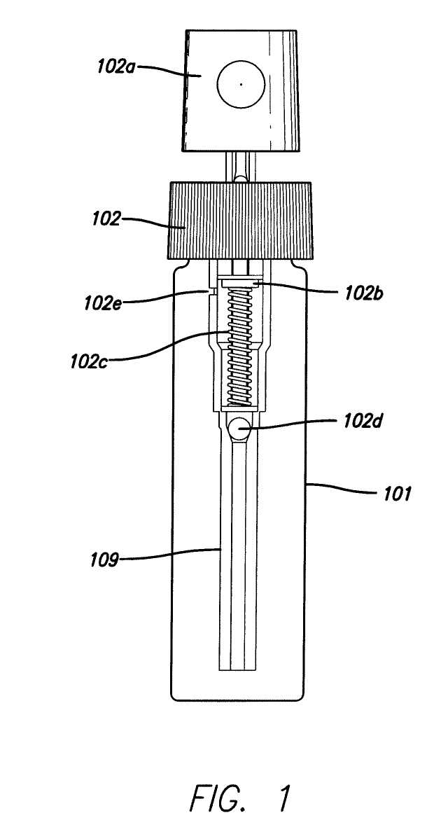

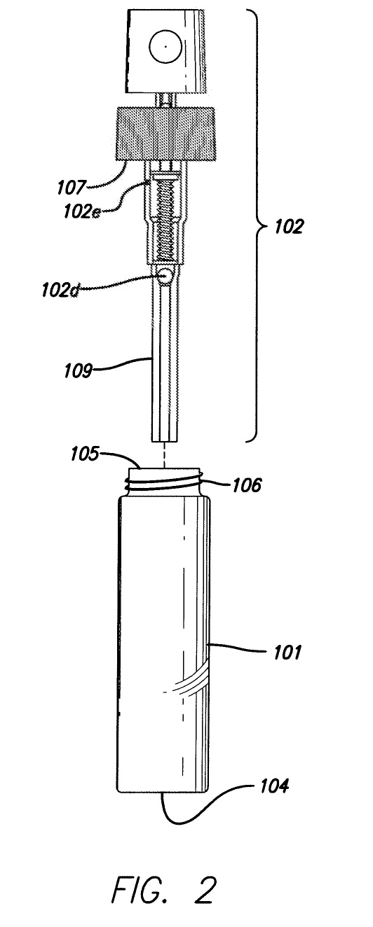

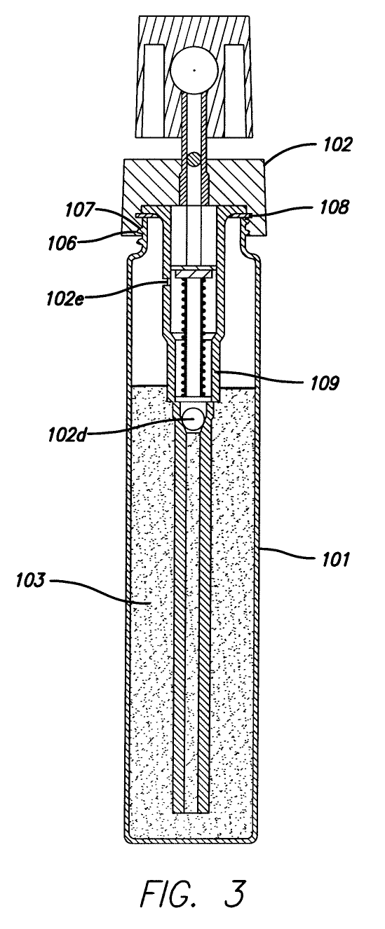

As illustrated in FIGS. 1-3, in one embodiment, the invention includes a vial 101 with a dispenser assembly 102. An agent 103 may be stored in the vial 101 and delivered by operation of the dispenser assembly 102.

In an embodiment of the present invention, the agent 103 contains a pharmaceutically active ingredient in a carrier. In another embodiment, the agent 103 is a composition to prevent and/or treat and/or reduce the risk of an infection. In another embodiment, the agent 103 is antimicrobial, antiviral, antimycobacterial, antiftungal and/or sporicidal. In another embodiment, the agent 103 is substantially non-irritating to the eyes, ears, mouth, nose, wounds, mucous membranes and/or non-mucous membranes of a human subject. In another embodiment, the agent 103 is substantially alcohol-free. In another embodiment, the agent 103 contains a solution of oxychlorine compounds. In another embodiment, the agent 103 contains about 99.99% oxidized water, sodium hypochlorite (NaOCl), hypochlorous acid (HOCl) and sodium chloride (NaCl). In another embodiment, the agent 103 is Microcyn® OTC Wound Care (available from Oculus Innovative Sciences, Inc.). In another embodiment, the agent is among the products described in U.S. patent application publication Nos. 2005/0196462, 2006/0235350, 2006/0241546, 2006/0253060, 2006/0272954, 2007/0173460, 2007/0173755, 2007/0196357 or 2007/0196434, each of which is incorporated by reference herein in its entirety as if fully set forth.

The vial 101 illustratively depicted in FIGS. 1-3 is generally cylindrical along its axis, with a closed base 104 and an open top 105. External screw threading 106 surrounds the open top 105, to enable the mechanical interaction of the vial 101 with corresponding internal screw threading 107 on the dispenser assembly 102. The mechanical interaction of the external screw threading 106 on the vial 101 with the internal screw threading 107 on the dispenser assembly 102 may create a substantially fluid-tight seal between these elements. A washer 108 may be included between the open top 105 and the dispenser assembly 102 to provide an additional (and in some cases less permeable) fluid seal. The washer may be constructed from any suitable material, as will be readily appreciated by those of skill in the art, such as, alone or in combination, an elastomeric material, rubber, silicone, plastic, polytetrafluoroethylene (“PTFE;” available under the trade name Teflon® from DuPont Corporation), the fluoropolymer PFA (e.g., perfluoroalkoxy polymer resin; also available under the trade name Teflon® from DuPont Corporation), the Dyneon™ TFM™ range of chemically-modified PTFE (available from 3M), and any number of other materials that are substantially inert or non-reactive with regard to the agent 103. In alternate embodiments of the present invention, different mechanisms may be used to affix the dispenser assembly 102 to the vial 101; for example, but in no way limited to, a snap fitting, a pressure fitting, a heat seal, a chemical adhesive or any number of other mechanical or chemical mechanisms readily known to those of skill in the art.

In alternate embodiments of the present invention, the vial 101 may be selected from any of a variety of shapes, such as, alone or in combination, cylindrical, rectangular, spheroid or any other desirable geometric configuration. In alternate embodiments, the vial 101 may have a shape that remains consistent or changes in cross-section along its axis, such as circular, elliptical, square, rectangular, rhomboid, trapezoidal, triangular, pentagonal, hexagonal, octagonal, and so on. As noted above, as illustratively depicted in FIGS. 1-3, the vial 101 is generally cylindrical and thus retains a generally circular shape along the entirety of its axis.

The vial 101 may be constructed from any suitable material. The material may be selected based upon, among other things, the chemical properties of the agent 103 that is to be contained in the vial 101. Specifically, it may be desirable to select a material that does not have a deleterious impact on the chemical composition, medicinal efficacy, stability or other properties of the agent 103; particularly insofar as the agent 103 is to be stored in the vial 101 for any appreciable amount of time, as may be necessitated by the intended use of the present invention. By way of example, in some embodiments the vial 101 may be constructed from various types of plastic, metal, stainless steel, glass (e.g., borosilicate glass), PTFE, the fluoropolymer PFA (e.g., perfluoroalkoxy polymer resin), the Dyneon™ TFM™ range of chemically-modified PTFE, and any number of other materials that are substantially inert or non-reactive with regard to the agent 103. As will be readily appreciated by those of skill in the art, a variety of different materials may be used to construct the vial 101 and may be desirable based on the selection of various agents 103, and such materials are all contemplated as being within the scope of the present invention and can be readily used in alternate embodiments thereof without undue experimentation.

The dispenser assembly 102 may be selected from any apparatus useful to dispense the agent 103 from the vial 101. Those of skill in the art will recognize numerous such apparatuses, which may include, but are no way limited to, pump sprayers, squeeze tops (e.g., those akin to the end of a conventional tube of toothpaste), aerosol sprayers, eye-droppers and applicator pads. As illustratively depicted in FIG. 1, in one embodiment of the present invention, the dispenser assembly 102 is a pump sprayer, and is configured with conventional components, including a spray head 102a, a plunger 102b, a spring 102c, a ball 102d and a hole 102e. The components may be constructed from any desirable materials, which, in some embodiments, are inert with respect to the agent 103. The ball 102d may be constructed from any desirable material, which, in one embodiment, is stainless steel. Because certain components of the dispenser assembly 102 only come into contact with the agent 103 upon actuation (i.e., when a user dispenses the agent 103 from the vial 101), such components may need not be constructed of materials that are inert with respect to the agent. In those embodiments of the invention where the dispenser assembly 102 is a pump sprayer, the components that do not come into contact with the agent 103 until actuation may include the spray head 102a, plunger 102b, spring 102c and/or ball 102d.

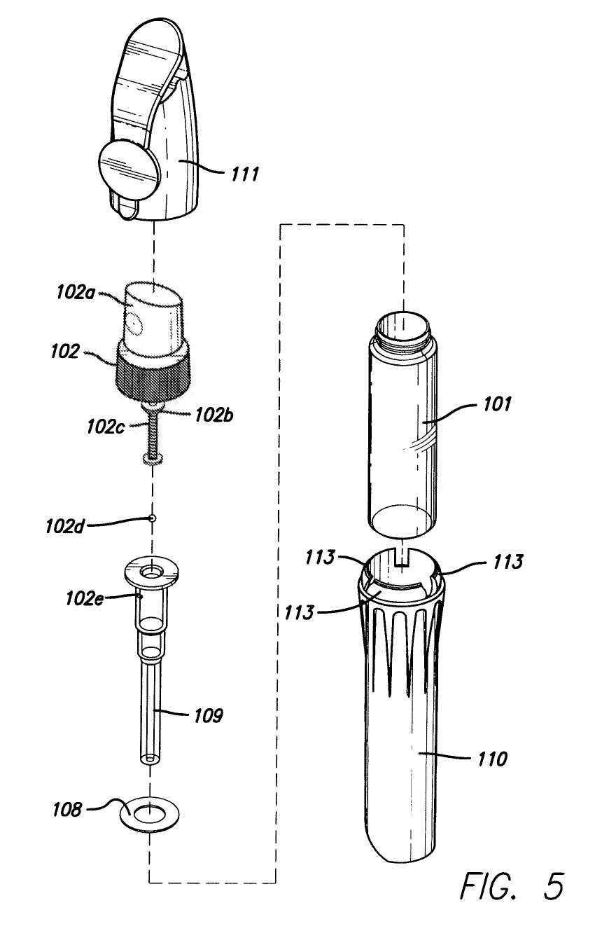

Certain components of the dispenser assembly 102 may remain in fluid communication with the agent 103, irrespective of device actuation (e.g., during such time as the agent 103 is stored in the vial 101). For instance, in those embodiments of the present invention when the dispenser assembly 102 is a pump sprayer (as depicted in FIGS. 1-3 and 5), an insert portion 109 thereof may extend axially from the portion of the pump sprayer that remains external to the vial 101, into the interior of the vial 101 where the agent 103 is contained. In certain embodiments of the present invention it may be desirable for the insert portion 109 to be constructed of a material that does not have a deleterious impact on the chemical composition, medicinal efficacy or other properties of the agent 103. Therefore, in particular embodiments of the present invention, the insert portion 109 may be constructed from various types of glass (e.g., borosilicate glass), PTFE, the fluoropolymer PFA (e.g., perfluoroalkoxy polymer resin), the Dyneon™ TFM™ range of chemically-modified PTFE, and any number of other materials that are substantially inert or non-reactive with regard to the agent 103. The insert portion 109 may be substantially rigid and configured to reach a region of the vial 101 near its closed base 104, such that most, if not substantially all of the agent 103 contained in the vial 101 may be dispensed through operation of the dispenser assembly 102. In various embodiments of the present invention, the radial dimensions of the insert portion 109 may vary along its length, or, alternatively, remain substantially constant. In the embodiment illustratively pictured in FIGS. 1-3 and 5, the insert portion 109 includes exterior radii of different dimensions along its length, as well as interior radii of increasingly narrow dimension from the end of the insert portion 109 nearest the open top 105 of the vial 101 to its closed base 104. Among other things, such a configuration may serve to accommodate the various components of the dispenser assembly 102 and/or minimize the amount of material needed to manufacture the insert portion 109. In an embodiment, the insert portion 109 is a unitary item, rather than several segments that are manufactured separately and adhered or otherwise affixed to one another; although both alternatives are envisioned as being within the scope of the instant invention.

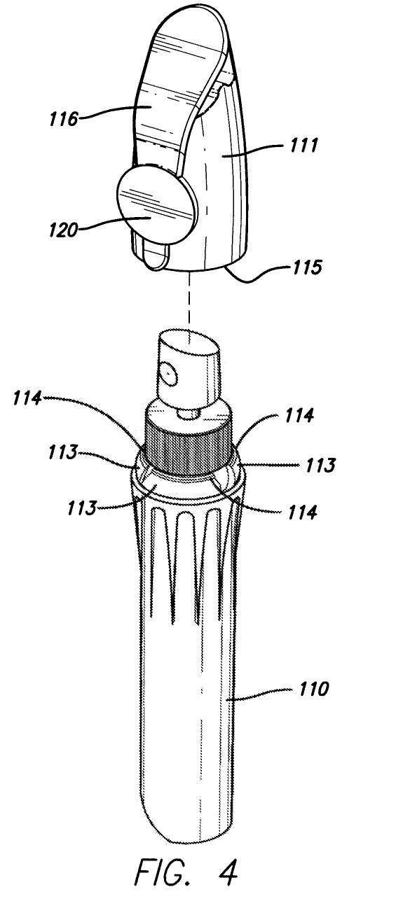

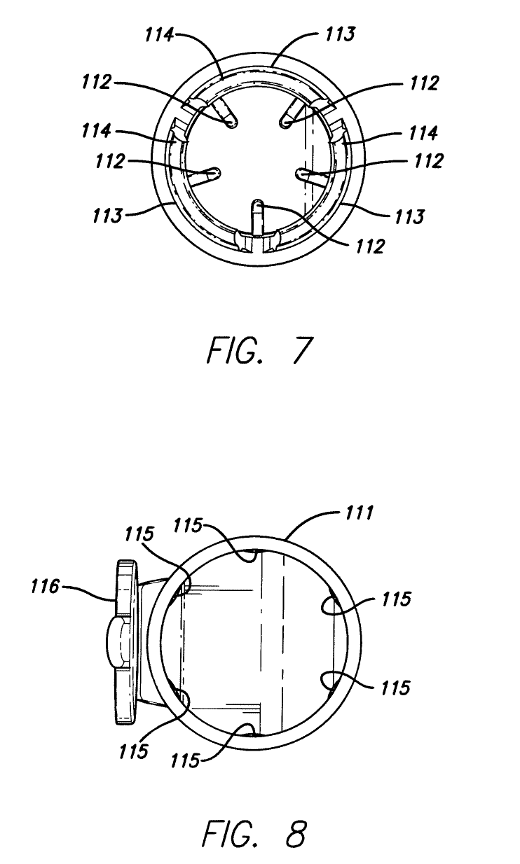

In another embodiment of the present invention, as depicted in FIGS. 4-7, the vial 101 and dispenser assembly 102 may be fitted within a barrel 110 and cap 111. The vial 101 may be stabilized within the barrel by a series of support elements 112 in mechanical contact with the closed base 104 on one end, and a series of flanges 113 that grasp the vial about its open top 105 beneath the external screw threading 106. The series of flanges 113 may be configured to separate in a radial direction from one another to allow the vial 101 to be inserted into the barrel 110. Once inserted, the flanges 113 may provide the aforementioned grasping force to retain the vial 101 within the barrel 110, such that it cannot be easily removed therefrom or slide out of the barrel 110 during normal operation. In an alternate embodiment, the flanges 113 exert substantially minimal force on said vial 101, but instead retain the vial 101 within the barrel 110 by virtue of having a circumference generally smaller than that of the outer surface of the vial 101; such that the vial 101 cannot slide through the flanges 113 without substantial force.

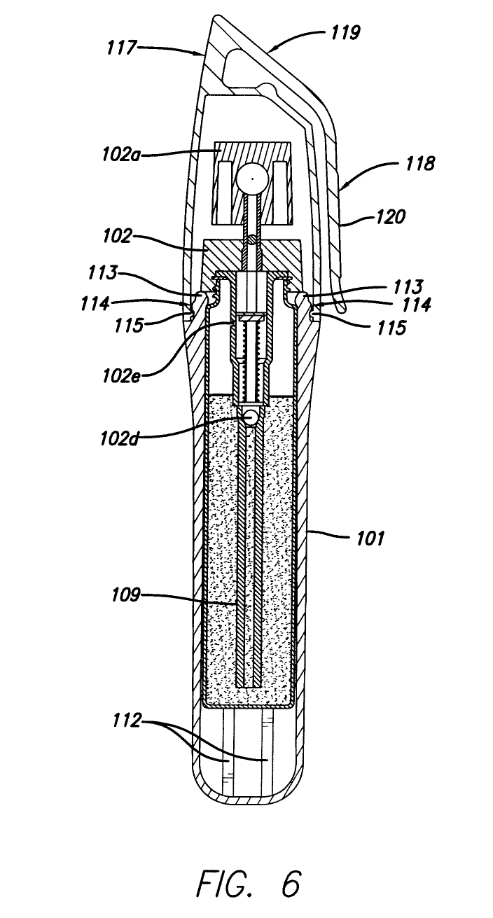

The flanges 113 may have locking elements 114 on their exterior surface configured to mechanically interact with receiving elements 115 on the interior surface of the cap 111 (FIG. 8). The mechanical interaction of the locking elements 114 with the receiving elements 115 may keep the cap 111 and barrel 110 affixed to one another until such time as the cap 111 is intentionally removed from the barrel 110 by a user. As illustrated in FIG. 6, when the cap 111 and barrel 110 are locked together, the dispenser assembly 102 is protected from inadvertent use.

In another embodiment, the cap 111 may include a tab 116, configured to allow a user to easily remove the cap 111 from the barrel 110 by dislodging the locking elements 114 from the receiving elements 115. As illustrated in FIG. 6, in one embodiment, the tab 116 includes a forward portion 117 affixed to one side of the cap 111, a rear portion 118 configured to receive a mechanical force from a user to separate the cap 111 from the barrel 110, and an intermediate portion 119 therebetween. The angle between the forward portion 117 and the axis of the cap 111 may be from about 0° to about 10°; the angle between the intermediate portion 119 and the axis of the cap 111 may be from about 45° to about 55°, and in one embodiment, about 50°; and the angle between the rear portion 118 and the axis of the cap 111 may be from about 0° to about 8°. The rear portion 118 may include a surface 120 configured to receive the finger of a user, the generally radial force (i.e., including, at most, a minimal longitudinal component) of which upon the surface 120 may dislodge the cap 111 from the barrel 110.

### EXAMPLE 1

### Elimination of Microorganisms, Reduction of Bacterial and/or Viral Titers, Germ-Killing and/or Antifungal/Sporicidal Activity on a Surface Area of a Human Subject

A device as described herein is provided, including a glass vial with a pump sprayer, wherein the elements of the pump sprayer that are in direct contact with the agent contained in the device irrespective of device actuation are made from an inert material with respect to the agent. The glass vial includes a volume of an agent containing about 99.99% oxidized water, sodium hypochlorite (NaOCl), hypochlorous acid (HOCl) and sodium chloride (NaCl). The glass vial and pump sprayer are fitted within a plastic barrel and cap assembly; the cap includes a tab with a surface configured to receive a user's finger.

A user exerts a generally radial force on the tab surface; thereby releasing the cap from the barrel. The user then sprays a quantity of the agent onto a wound, into his eyes, into his ears, into his mouth and/or onto any additional surfaces of his body after being contacted by a substance believed to contain pathogens. The agent prevents, treats and/or reduces the risk of an infection on the surfaces to which it is applied.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s). Furthermore, no limitations are intended to the details of construction or design herein shown other than as described in the claims below. It is, therefore, evident that the particular embodiments disclosed above may be altered or modified and all such variations are considered within the scope and spirit of the invention. Accordingly, the protection sought herein is as set forth in the claims below.

What is claimed is:

1. An apparatus, comprising:
a vial to contain an agent; and
a dispenser assembly to dispense said agent from said vial, said dispenser assembly mechanically affixed to said vial, and said dispenser assembly including elements that come into contact with said agent irrespective of actuation,
wherein said vial and said elements that come into contact with said agent irrespective of actuation consist essentially of materials that are inert or non-reactive with the agent.

2. The apparatus of claim 1, further comprising a barrel and cap, within which said vial and dispenser assembly are fitted.

3. The apparatus of claim 1, further comprising a quantity of an agent.

4. The apparatus of claim 2, wherein said agent is a composition to prevent and/or treat and/or reduce the risk of an infection.

5. The apparatus of claim 2, wherein said agent contains about 99.99% oxidized water, sodium hypochlorite (NaOCl), hypochlorous acid (HOCl) and sodium chloride (NaCl).

6. The apparatus of claim 1, wherein said barrel further comprises a series of flanges to retain said vial within said barrel.

7. The apparatus of claim 6, wherein said series of flanges are configured to separate from one another to allow said vial to be inserted into said barrel, and thereafter substantially assume an initial shape that retains said vial within said barrel.

8. The apparatus of claim 1, wherein said barrel further comprises at least one support element in mechanical contact with said vial.

9. The apparatus of claim 1, wherein said dispenser assembly is a pump sprayer.

10. The apparatus of claim 9, wherein said pump sprayer further comprises an insert portion extending axially from the portion of the pump sprayer that remains external to the vial, into an interior of the vial.

11. The apparatus of claim 10, wherein said insert portion is a unitary item.

12. The apparatus of claim 10, wherein said vial and said insert portion are each constructed of materials independently selected from the group consisting of glass, borosilicate glass, PTFE, PFA, chemically-modified PTFE, inert materials and combinations thereof.

13. The apparatus of claim 1, further comprising a washer positioned between said vial and said dispenser assembly to provide and/or enhance a fluid seal therebetween.

14. The apparatus of claim 13, wherein said washer is constructed of a material selected from the group consisting of PTFE, PFA, chemically-modified PTFE, inert materials and combinations thereof.

15. The apparatus of claim 1, wherein said vial and said dispenser assembly are affixed to one another with a mechanism selected from the group consisting of screw threading, a snap fitting, a pressure fitting, a heat seal, a chemical adhesive and combinations thereof.

16. The apparatus of claim 6, wherein said series of flanges each comprise a locking element configured to mechanically interact with at least one receiving element configured on an interior surface of said cap.

17. The apparatus of claim 16, wherein said cap further comprises a tab configured to allow a user to remove said cap from said barrel by dislodging said locking elements from said at least one receiving element.

18. The apparatus of claim 17, wherein said tab comprises a forward portion affixed to a side of said cap, a rear portion configured to receive a mechanical force from the user to separate said cap from said barrel, and an intermediate portion therebetween.

19. The apparatus of claim 18, wherein the angle between said forward portion and an axis of said cap is from about 0° to about 10°, the angle between said intermediate portion and said axis of said cap is from about 45° to about 55°, and the angle between said rear portion and said axis of said cap is from about 0° to about 8°.

20. The apparatus of claim 18, wherein said rear portion comprises a surface configured to receive a finger of the user and, upon application of a generally radial force thereupon, dislodge said cap from said barrel.

21. A method of preventing and/or treating and/or reducing the risk of an infection in a subject in need thereof, comprising:
providing an apparatus, comprising
a vial containing a composition to prevent and/or treat and/or reduce the risk of an infection,
a dispenser assembly to dispense said composition from said vial, said dispenser assembly mechanically affixed to said vial, and said dispenser assembly including elements that come into contact with said composition irrespective of actuation, and
a barrel and cap, within which said vial and said dispenser assembly are fitted,
wherein said vial and said elements that come into contact with said composition irrespective of actuation consist essentially of materials that are inert or non-reactive with the composition;
removing said cap from said barrel; and
dispensing said composition onto a surface of said subject to prevent and/or treat and/or reduce the risk of said infection.

22. The method of claim 21, wherein said composition contains about 99.99% oxidized water, sodium hypochlorite (NaOCl), hypochlorous acid (HOCl) and sodium chloride (NaCl).

23. The method of claim 21, wherein said barrel further comprises a series of flanges to retain said vial within said barrel, and said series of flanges are configured to separate from one another to allow said vial to be inserted into said barrel, and thereafter substantially assume an initial shape that retains said vial within said barrel.

24. The method of claim 21, wherein said dispenser assembly is a pump sprayer, comprising an insert portion extending axially from the portion of the pump sprayer that remains external to the vial, into an interior of the vial, and wherein said insert portion is a unitary item.

25. The method of claim 24, wherein said vial and said insert portion are each constructed of materials independently selected from the group consisting of glass, borosilicate glass, PTFE, PFA, chemically-modified PTFE, inert materials and combinations thereof.
